(19) United States
(12) Plant Patent Application Publication
(10) Pub. No.: US 2018/0077842 P1
(43) Pub. Date: Mar. 15, 2018

(54) Peperomia plant named 'Little Toscani'

(50) ;

(71) Applicant: Obed Jacob Smit, Sappemeer (NL)

(72) Inventor: Obed Jacob Smit, Sappemeer (NL)

(21) Appl. No.: 15/330,401

(22) Filed: Sep. 15, 2016

(51) Int. Cl. A01H 5/12

(52) U.S. Cl. PLT/373

(57) ABSTRACT

A new cultivar of Peperomia plant named ‘Little Toscani’ that is characterized by a rounded shape, a compact habit and small leaves that have a grey-green colored pattern on the upper surface.

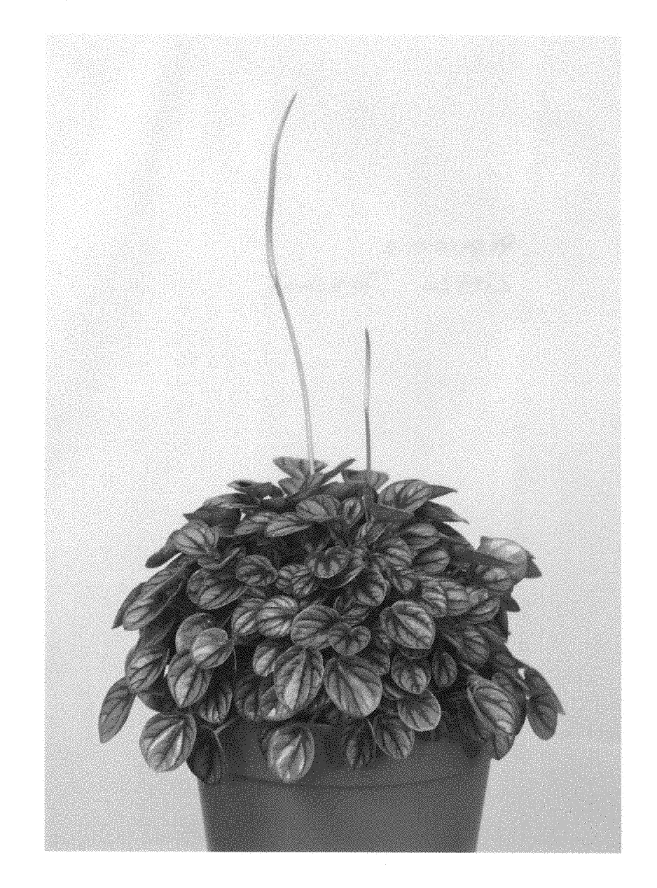

Botanical classification: Peperomia hybrid.

Variety denomination: ‘Little Toscani’.

### BACKGROUND OF THE INVENTION

The present invention relates to a new and distinct cultivar of Peperomia plant botanically known as Peperomia hybrid and hereinafter referred to by the cultivar name ‘Little Toscani’.

‘Little Toscani’ originated from the crossing of the female or seed parent, an unnamed Peperomia peruviana cultivar and the male or pollen parent, an unnamed Peperomia caperata cultivar. The crossing was conducted in 2013 in Sappemeer, Netherlands. The resulting seeds were subsequently planted and grown. The cultivar ‘Little Toscani’ was selected by the inventor in 2014 in a controlled environment as a single plant within the progeny of the stated cross in a cultivated area of Sappemeer, Netherlands.

Asexual reproduction of the new cultivar ‘Little Toscani’ first occurred by leaf cuttings in 2014 in Sappemeer, Netherlands. Since that time, under careful observation, the unique characteristics of the new cultivar have been uniform, stable and reproduced true to type in successive generations of asexual reproduction.

### SUMMARY OF THE INVENTION

The following represent the distinguishing characteristics of the new Peperomia cultivar ‘Little Toscani’. These traits in combination distinguish ‘Little Toscani’ as a new and distinct cultivar apart from other existing varieties of Peperomia known by the inventor.

1. Peperomia ‘Little Toscani’ exhibits a rounded shape and a compact habit.
2. Peperomia ‘Little Toscani’ exhibits small leaves that have a grey-green colored pattern on the upper surface.

The closest comparison cultivars are Peperomia ‘Eden Rosso’ (U.S. Plant Pat. No. 24,379) and Peperomia ‘Napoli Nights’ (co-pending U.S. Plant patent application). ‘Little Toscani’ is distinguishable from ‘Eden Rosso’ by the following characteristics:

1. Peperomia ‘Little Toscani’ exhibits leaves having an upper surface with a grey-green colored pattern. In comparison, the upper leaf surface of ‘Eden Rosso’ is green.
2. Peperomia ‘Little Toscani’ exhibits leaves having a lower surface that is light green. In comparison, the leaf lower surface of ‘Eden Rosso’ is red.

‘Little Toscani’ is distinguishable from ‘Napoli Nights’ by the following characteristics:

1. Peperomia ‘Little Toscani’ exhibits smaller leaves than the leaves of ‘Napoli Nights’.
2. Peperomia ‘Little Toscani’ exhibits leaves having a lower surface that is light green. In comparison, the leaf lower surface of ‘Napoli Nights’ is pink-red.

‘Little Toscani’ is distinguishable from the female parent plant, by the following characteristics:

1. The leaves of ‘Little Toscani’ are smaller and more oval in shape than the leaves of the female parent plant.
2. The upper surface of the leaves of ‘Little Toscani’ exhibit a grey-green pattern. In comparison, the upper surface of the leaves of the female parent plant are green.

‘Little Toscani’ is distinguishable from the male parent plant by the following characteristics:

1. The leaves of ‘Little Toscani’ are smaller and more oval in shape than the leaves of the male parent plant.
2. ‘Little Toscani’ exhibits leaf veins that spread out across the leaf more than the leaf veins of the male parent plant.

### BRIEF DESCRIPTION OF THE DRAWING

The accompanying photograph illustrates the distinguishing traits of Peperomia ‘Little Toscani’. The photograph shows an overall view of a 35 week old plant. The photograph was taken using conventional techniques and although colors may appear different from actual colors due to light reflectance, it is as accurate as possible by conventional photographic techniques.

### BOTANICAL DESCRIPTION OF THE PLANT

The following is a detailed description of the new Peperomia cultivar named ‘Little Toscani’. Data was collected in Sappemeer, Netherlands from 35 week old plants grown in a glass greenhouse in 15 cm. diameter containers. The time of year was Winter and the temperature range was 18-25 degrees Centigrade during the day and 15-18 degrees Centigrade at night. The light level was natural light level. No photoperiodic treatments or growth retardants were used. Color determinations are in accordance with The Royal Horticultural Society Colour Chart 2015 edition, except where general color terms of ordinary dictionary significance are used. The growing requirements are similar to the species. ‘Little Toscani’ has not been tested under all possible conditions and phenotypic differences may be observed with variations in environmental, climatic, and cultural conditions, however, without any variance in genotype.
Botanical classification: Peperomia hybrid ‘Little Toscani’.
Annual or perennial: Perennial.
Parentage: ‘Little Toscani’ is a hybrid of the female parent, an unnamed Peperomia peruviana cultivar and the male parent an unnamed Peperomia caperata cultivar.
Plant type: Potted plant.
Growth habit: Rosette.
Plant shape: Flattened globe shaped.
Suitable container size: 7 cm. pots or larger.
Plant height: 10.9 cm.
Plant width: 24.2 cm.
Vigor: Low.
Low temperature tolerance: 5° Centigrade.
High temperature tolerance: 40° Centigrade.
Propagation: Leaf cuttings.
Time to initiate roots (summer): 10 days at 18 to 20° C.
Time to initiate roots (winter): 14 days at 18 to 20° C.
Time to produce a rooted cutting (summer): 80 days at 18 to 20° C.
Time to produce a rooted cutting (winter): 100 days at 18 to 20° C.
Growth rate: Moderate.
Crop time: Approximately 30 to 35 weeks in Sappemeer, Netherlands.
Root system: Fibrous.
Plant fragrance: None.
Stem:
   Branching habit.—Short thick stems branching into rosettes of leaves.
   Number of main stems per plant.—Average 13.
   Number of lateral branches per plant.—Average 52.
   Lateral branch dimensions.—1.5 cm. in length and 0.6 cm. in width.
   Internode length.—0.075 cm.
   Stem shape.—Rounded.
   Stem texture.—Smooth.
   Stem pubescence.—Absent.
   Stem angle.—5 degrees.
   Stem strength.—Strong.
   Stem color (young).—146D.
   Stem color (mature).—145B, tinged 182B.
   Internode color.—145B, tinged 182B.
Foliage:
   Leaf arrangement.—Alternate.
   Compound or single.—Single.
   Quantity of leaves per lateral branch.—Average 20.
   Leaf shape.—Broad ovate.
   Leaf aspect.—Slightly convex.
   Leaf apex.—Rounded.
   Leaf base.—Truncate.
   Leaf dimensions.—2.5 cm. in length and 2.2 cm. in width.
   Texture.—Glabrous (both surfaces).
   Leaf luster.—Glossy (both surfaces).
   Pubescence.—Absent.
   Leaf margin.—Entire.
   Venation pattern.—Parallel, slightly furrowed.
   Young leaf color (upper surface).—191B, surrounding veins 147A.
   Young leaf color (lower surface).—148C to 148D, surrounding veins 148A.
   Mature leaf color (upper surface).—N189B to N189C.
   Mature leaf color (lower surface).—148B.
   Vein color (upper surface).—147A.
   Vein color (lower surface).—148A to 147B.
Petiole:
   Petiole dimensions.—6.2 cm. in length and 0.2 cm. in diameter.
   Petiole texture.—Glabrous.
   Petiole luster.—Slightly glossy.
   Petiole pubescence.—Absent.
   Petiole strength.—Low.
   Petiole color.—148D with fine stripes 182A.
Flower:
   Inflorescence type.—Axillary spike.
   Inflorescence dimensions.—9.1 cm in length and 0.35 cm in diameter.
   Quantity of flowers per inflorescence.—Average 700.
   Quantity of flowers and buds per plant.—Average 1500.
   Flowering habit.—Flowers occasionally occur on mature plants.
   Fragrance.—None.
   Bud length.—Average 0.2 mm.
   Bud diameter.—Average 0.2 mm.
   Bud shape.—Globose.
   Bud color.—144C.
   Flower type and form.—Flowers have no petals and consist of four stamens and a single pistil.
   Flower aspect.—Outward.
   Flower dimensions.—Average 0.4 mm. in diameter and 0.6 mm. in height.
   Flower longevity.—Approximately 1 month.
   Persistent or self-cleaning.—Self-cleaning.
Peduncle:
   Peduncle dimensions.—14.1 cm. in length and 0.2 cm. in diameter.
   Peduncle angle.—20 degrees from vertical.
   Peduncle strength.—Moderately weak.
   Peduncle texture.—Smooth.
   Peduncle luster.—Glossy.
   Peduncle color.—148D with fine stripes 182B.
Reproductive organs:
   Stamen number.—4.
   Anther shape.—Club shaped.
   Anther length.—0.1 mm.
   Anther color.—144C.
   Filament length.—0.1 mm.
   Filament color.—144C.
   Amount of pollen.—Moderate.
   Pollen color.—156D.
   Number of pistils.—1.
   Pistil length.—0.05 mm.
   Stigma shape.—Flattened, ovate.
   Stigma dimensions.—0.05 mm. in length and 0.2 mm. in diameter.
   Stigma color.—144C.
   Style.—None visible.
   Ovary color.—144C.
Fruit and seed: ‘Little Toscani’ has not produced fruit or seed to date.
Disease and pest resistance: Disease and pest resistance has not been observed.

1. A new and distinct variety of Peperomia plant named ‘Little Toscani’ as described and illustrated.
